(19) United States
(12) Patent Application Publication
(10) Pub. No.: US 2026/0316067 A1
(43) Pub. Date: Oct. 15, 2026

(54) Pharmaceutical Compounding System

(71) Applicant: Germfree Laboratories, LLC, Ormond Beach, FL (US)

(72) Inventors: Judson Whigham Baker, Ormond Beach, FL (US); Christopher Doane, Ormond Beach, FL (US); Carol A. Houts, Ormond Beach, FL (US)

(73) Assignee: Germfree Laboratories, LLC, Ormond Beach, FL (US)

(21) Appl. No.: 19/457,974

(22) Filed: Jan. 23, 2026

Related U.S. Application Data
(63) Continuation of application No. PCT/US24/39607, filed on Jul. 25, 2024.
(60) Provisional application No. 63/515,562, filed on Jul. 25, 2023.
(60) Provisional application No. 63/565,442, filed on Mar. 14, 2024.

(51) Int. Cl. B01L 1/02; G16H 20/10; G16H 40/63

(52) U.S. Cl. CPC B01L 1/025; G16H 20/10; G16H 40/63; B01L 2300/027; B01L 2300/0681

(57) ABSTRACT

A system for pharmaceutical compounding, includes a workspace surface having an outer boundary, a display embedded within the workspace surface, the display having a plurality of edges that are impermeably sealed between the workspace surface, a computing device providing a user interface to the display, an enclosed housing extending from the outer boundary of the workspace surface and having an opening for a user to access the workspace surface, and an air filtration system providing a negative pressure airflow within the enclosed housing. The display is flush with the workspace surface. The system may further include a gravimetric scale positioned on the workspace surface, a cabinet attached to a first side of the enclosed housing, a label printer coupled to the computing device and positioned within the cabinet, and/or a code scanner coupled to the computing device and positioned within the cabinet.

### BACKGROUND

Laminar flow hoods, biological safety cabinets, and/or gloveboxes are used in a variety of applications. Many processes (e.g., including pharmaceutical compounding) that require laminar flow hoods, biological safety cabinets, and/or gloveboxes are done manually, inevitably leading to human error in the processes. Indeed, manual pharmaceutical compounding cause over 1000 deaths annually due to human errors or contamination. Additional features and/or controls have been implemented to reduce human error, however, these additional features and/or controls clutter the workspace and can cause potential contamination (e.g., sterility failures) of the products (e.g., medical drugs) that are created. Therefore, these additional features and/or controls may lead to serious harm or even death to patients if the contamination is not discovered, or waste of expensive products if the contamination is discovered. Accordingly, there is a need for systems and methods that declutter the working environment in laminar flow hoods, biological safety cabinets, and/or gloveboxes and optimize positioning of safety controls and/or features within laminar flow hoods, biological safety cabinets, and/or gloveboxes.

### BRIEF SUMMARY

Laminar flow hoods, biological safety cabinets, and/or gloveboxes including automation for pharmacological and biosafety purposes are described herein. Advantageously, the disclosed laminar flow hoods, biological safety cabinets, and/or gloveboxes disclosed herein provide features that reduce and/or eliminate human error while not cluttering the workspace and the associated sterility issues, which can not only save lives but also save valuable ingredients for pharmaceutical compounding and other processes.

A system for pharmaceutical compounding, includes a workspace surface having an outer boundary, a display embedded within the workspace surface, the display having a plurality of edges that are impermeably sealed between the workspace surface, a computing device providing a user interface to the display, an enclosed housing extending from the outer boundary of the workspace surface and having an opening for a user to access the workspace surface, and an air filtration system providing a negative pressure airflow within the enclosed housing. The display is flush with the workspace surface.

In some cases, the system further includes a gravimetric scale positioned on the workspace surface. In some cases, the system further includes a cabinet attached to a first side of the enclosed housing. In some cases, the system further includes a label printer coupled to the computing device and positioned within the cabinet. In some cases, the system further includes a code scanner coupled to the computing device and positioned within the cabinet.

In some cases, the computing device includes a processor, a storage, and instructions stored in the storage that when executed by the processor, cause the system to: receive, via the user interface, a selection of a desired product, display, at the user interface, a list of ingredients for the desired product, receive, from a code scanner, a plurality of codes, each code of the plurality of codes indicating an ingredient type, confirm that the ingredient type for each received code of the plurality of codes matches a corresponding ingredient type from the list of ingredients, receive, from a preparation camera, an ingredient image or video of one or more ingredients of the list of ingredients in a syringe, vial or IV bag, receive, from a gravimetric scale, a weight measurement of the one or more ingredients of the list of ingredients in the syringe, vial or the IV bag, and confirm that the weight measurement of the one or more ingredients of the list of ingredients in the syringe or the IV bag matches a corresponding weight measurement for producing the desired product.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A illustrates a front view of a laminar flow hood.

FIG. 1B illustrates an angled view of a laminar flow hood.

FIG. 1C illustrates a view of an angled view of a cabinet in a closed position that is attached to a laminar flow hood.

FIG. 1D illustrates a view of an angled view of a cabinet in an open position that is attached to a laminar flow hood.

FIG. 1E illustrates a side view of a laminar flow hood with a cabinet in an open position.

FIG. 1F illustrates an angled view of a person accessing a workspace in a laminar flow hood.

FIG. 2 illustrates bottom view of a computing device mounted to a laminar flow hood

FIG. 3 illustrates a method of using a system for pharmaceutical compounding.

FIG. 4 illustrates a block diagram of components of a computing device.

### DETAILED DESCRIPTION

Laminar flow hoods, biological safety cabinets, and/or gloveboxes including automation for pharmacological and biosafety purposes are described herein. Advantageously, the disclosed laminar flow hoods, biological safety cabinets, and/or gloveboxes disclosed herein provide features that reduce and/or eliminate human error while not cluttering the workspace and the associated sterility issues, which can not only save lives but also save valuable ingredients for pharmaceutical compounding and other processes.

Although the Figures described herein refer to a laminar flow hood, the features described with respect to the laminar flow hood may also be applied to a biological safety cabinet and/or glovebox without deviating from the inventive concept.

In some cases, pharmaceutical compounding or drug compounding refers to processes for mixing solutions, medicines, compounds, and/or drugs into one or more intravenous (IV) bags, vials, and/or syringes.

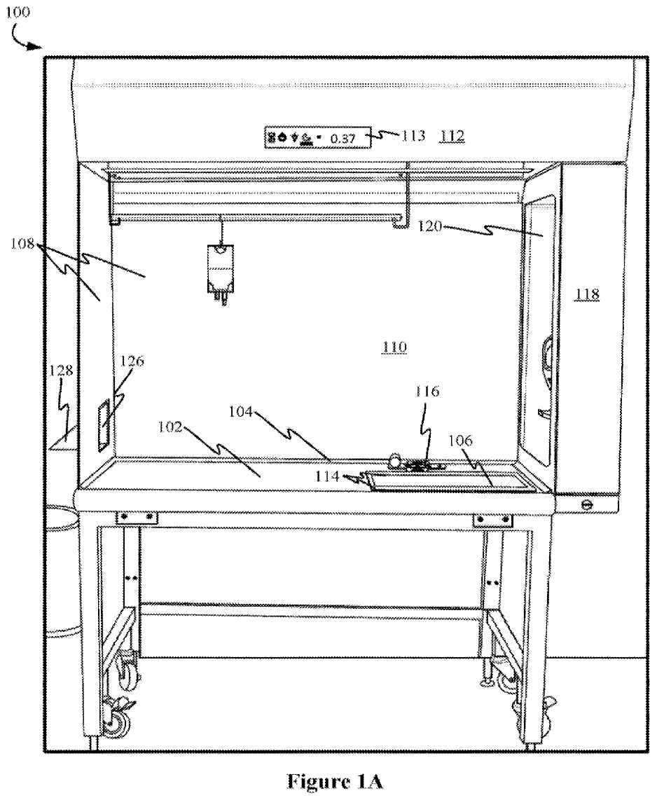

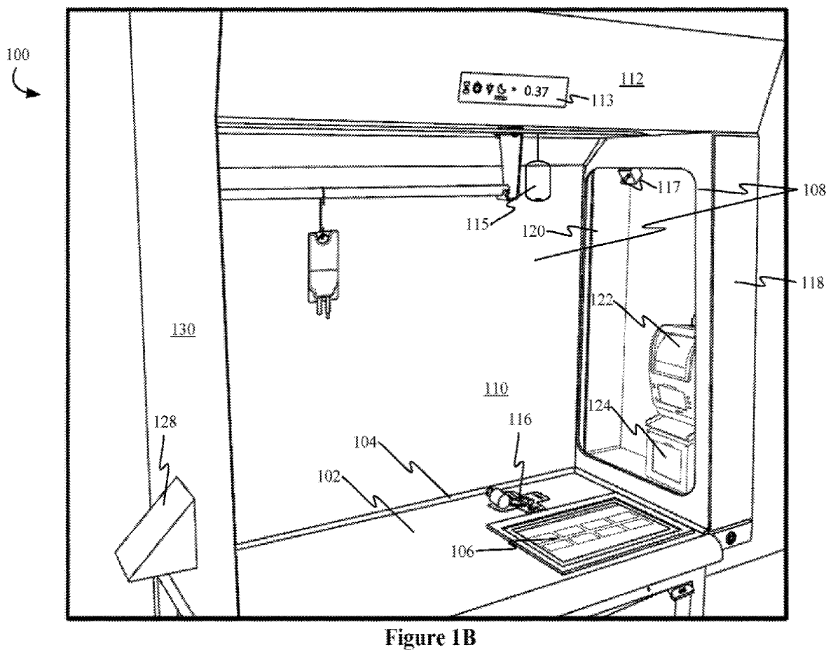

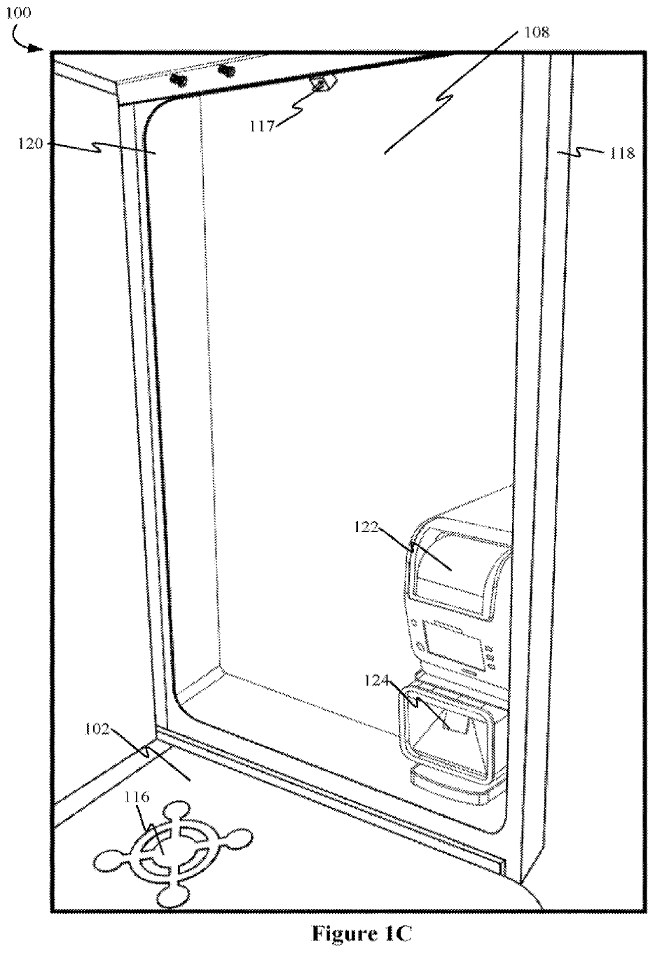

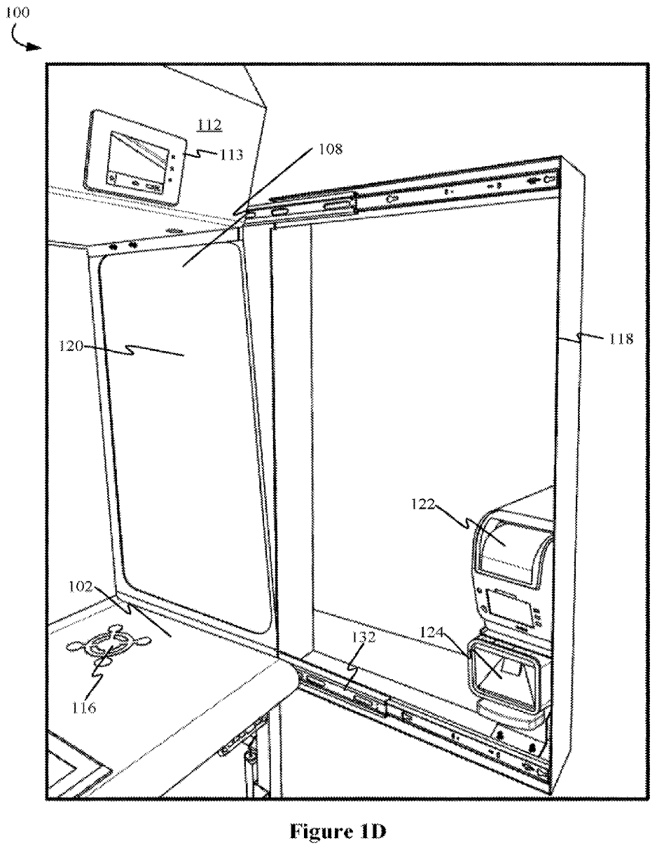

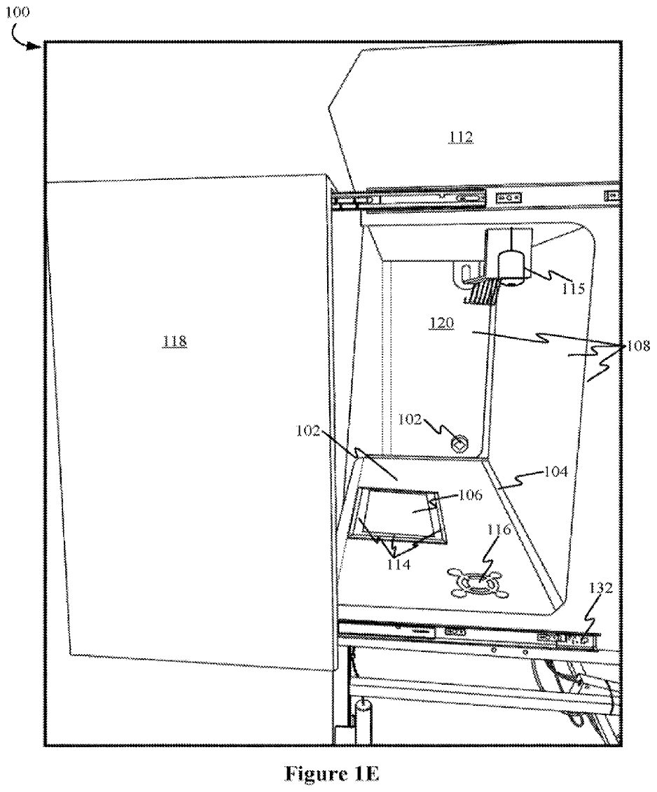

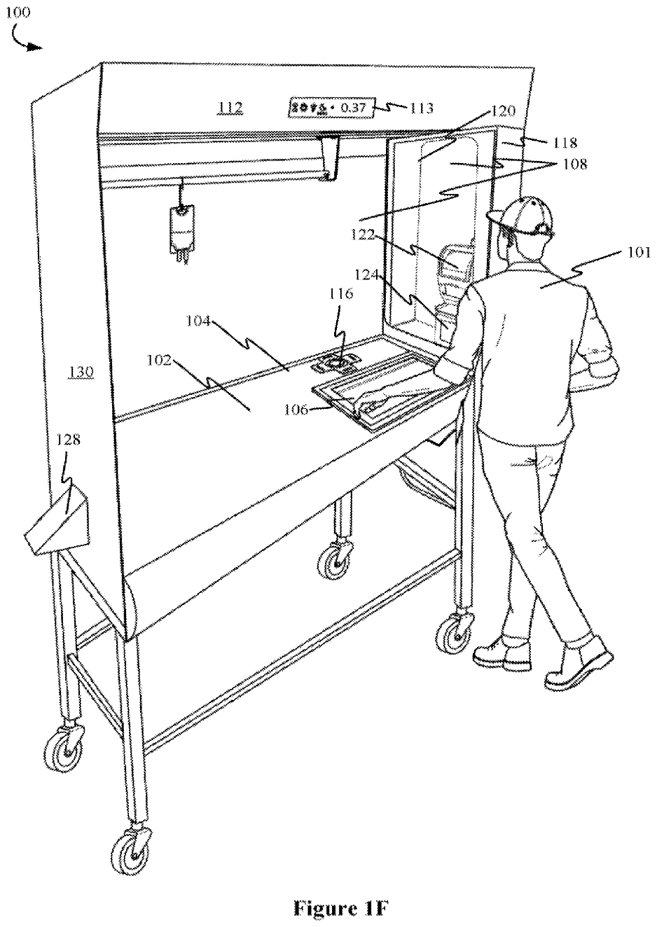

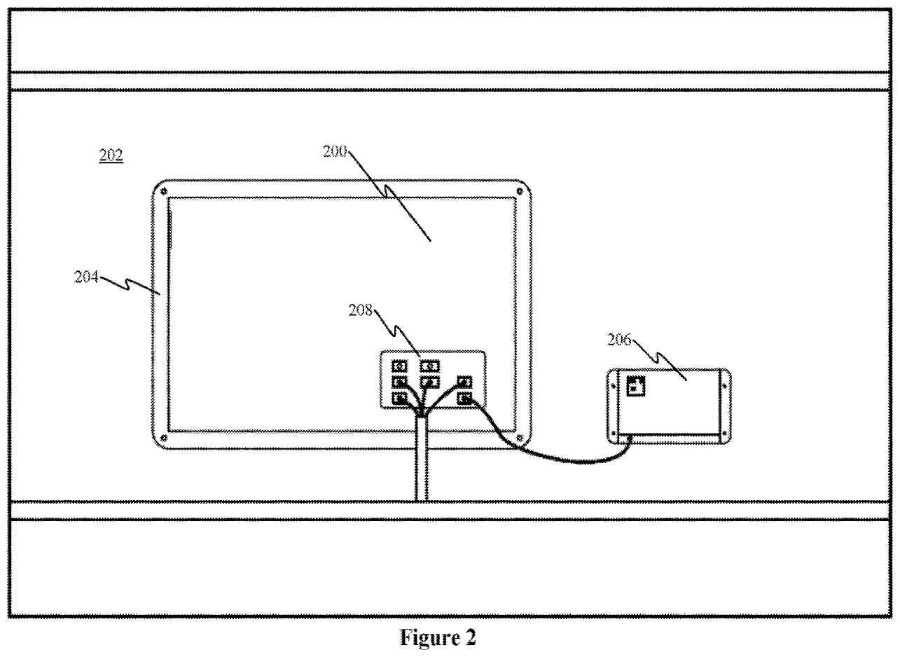

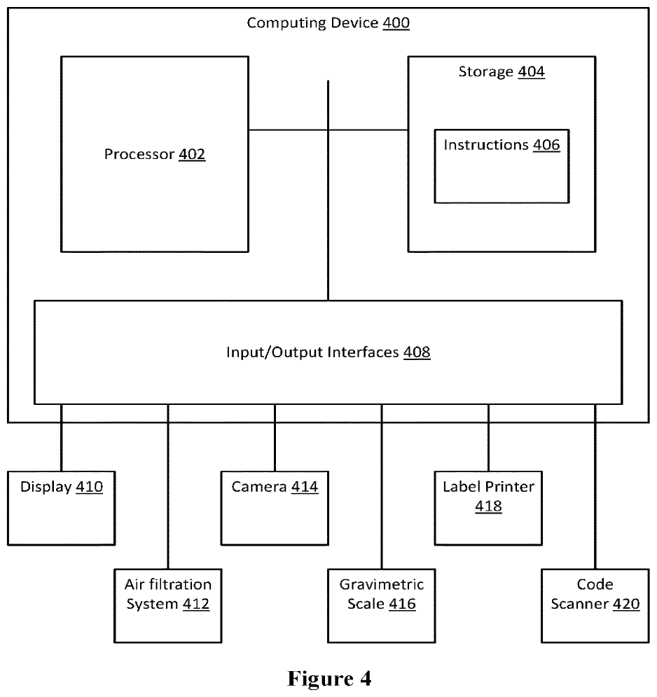

FIG. 1A illustrates a front view of a laminar flow hood. FIG. 1B illustrates an angled view of a laminar flow hood. Figure 1C illustrates a view of an angled view of a cabinet in a closed position that is attached to a laminar flow hood. FIG. 1D illustrates a view of an angled view of a cabinet in an open position that is attached to a laminar flow hood. FIG. 1E illustrates a side view of a laminar flow hood with a cabinet in an open position. FIG. 1F illustrates an angled view of a person accessing a workspace in a laminar flow hood. Referring to FIGS. 1A-1F, a laminar flow hood 100 for pharmaceutical compounding includes a workspace surface 102 having an outer boundary 104, a display 106 embedded within the workspace surface 102, a computing device (e.g., as illustrated with respect to FIGS. 2 and 4) providing a user interface to the display 106, an enclosed housing 108 extending from the outer boundary 104 of the workspace surface 102 and having an opening 110 for a user 101 to access the workspace surface 102, and an air filtration system embedded in an overhead enclosure 112 providing a negative pressure airflow within the enclosed housing 108. The display 106 includes a plurality of edges 114 that are impermeably sealed between the workspace surface 102. The display 106 is flush with the workspace surface 102.

In some cases, the enclosed housing 108 is impermeably sealed with the outer boundary 104 of the workspace surface 102 so that no air, liquid, and/or matter can pass through. In some cases, the outer boundary 104 is coved between the workspace surface 102 and the enclosed housing 108. In some cases, the workspace surface 102 is also impermeably sealed so that no air, liquid, and/or matter can pass through. In some cases, the air filtration system comprises high efficiency particulate (HEPA) filters. In some cases, the air filtration system providing the negative pressure airflow to the enclosed housing 108 also introduces at least a portion of the air removed from the enclosed housing 108 back to the enclosed housing 108 after the air has been filtered for particulates. In some cases, the overhead enclosure 112 includes a settings interface 113 for displaying and/or adjusting settings to features of the laminar flow hood.

In some cases, the laminar flow hood 100 further includes a gravimetric scale 116 positioned on the workspace surface 102. The gravimetric scale 116 can include a tray that is above and/or flush with the workspace surface 102 and a load cell positioned and/or mounted underneath the tray and the workspace surface 102. In some cases, the tray can be removed for cleaning purposes. A syringe and/or IV bag (e.g., containing one or more ingredients and/or a desired product) can be placed on the gravimetric scale 116 for gravimetric measurement and a preparation camera can capture pictures of and/or record video of that process.

In some cases, the laminar flow hood 100 further includes a preparation camera 115 positioned to capture images or video of the gravimetric scale 116 and/or workspace surface 102. For example, the preparation camera 115 may be positioned anywhere within the enclosed housing 108. In some cases, the preparation camera 115 is positioned within a cabinet 118. In some cases, the laminar flow hood 100 further includes a foot pedal coupled to the preparation camera 115 and/or the computing device. The foot pedal is configured to cause the preparation camera and/or a field-of-view camera 117 to capture an image or video of the gravimetric scale 116 when engaged (e.g., pressed) by the user 101 of the laminar flow hood 100. In some cases, a user interface of the display 106 may receive input from a user and cause the preparation camera 115 and/or the field-of-view camera 117 to capture a picture(s) or video. In some cases, the laminar flow hood 100 further includes a field-of-view camera 117 positioned to capture images or video of an entirety of the workspace surface 102 and/or entire area within the enclosed housing 108, including the actions of a user 101.

The one or more cameras (e.g., preparation camera 115 and/or field-of-view camera 117) can be mounted within and/or directly to the enclosed housing 108 of the laminar flow hood 100. An outer surface (e.g., an exterior lens) of the one or more cameras can be flush with the enclosed housing 108 of the laminar flow hood 100. The preparation camera 115 can be focused on the gravimetric scale 116 and/or workspace surface 102 and capture pictures and/or video of the gravimetric scale 116 and actions performed using the gravimetric scale 116 and/or workspace surface 102 (e.g., compounding processes). The preparation camera 115 can be mounted directly above the gravimetric scale 116. The field-of-view camera 117 can capture pictures and/or video of the entire workspace surface 102 and/or entire area within the enclosed housing 108 as well as processes performed by a user 101 on the workspace surface 102 and/or area within the enclosed housing 108. In some cases, the preparation camera 115 and/or the field-of-view camera 117 include a housing that is made of medical grade stainless steel or other similar material that can be cleaned and/or sanitized with, for example, cleaning solutions and/or ultraviolet light without being damaged.

In some cases, the laminar flow hood 100 further includes a cabinet 118 attached to a first side 120 of the enclosed housing 108. In some cases, the display 106 is a touchscreen. In some cases, the touchscreen includes a protective surface that can be cleaned and/or sanitized with, for example, cleaning solutions and/or ultraviolet light without being damaged. In some cases, the enclosed housing 108 is made of medical grade plexiglass and/or medical grade stainless steel. For example, a portion of the first side 120 of the enclosed housing 108 that is coupled to the cabinet 118 can be made of medical grade plexiglass with the rest of the enclosed housing made of medical grade stainless steel. In some cases, the workspace surface 102 is made of medical grade plexiglass and/or medical grade stainless steel. For example, the display 106 can be made of medical grade plexiglass and the workspace surface 102 surrounding the display 106 can be made of medical grade stainless steel.

The cabinet 118 may be attached to the first side 120 of the laminar flow hood 100. When in a closed position (e.g., as illustrated in FIG. 1C), the cabinet 118 is positioned behind medical grade plexiglass of the enclosed housing 108. When in the closed position, no particulates can enter the cabinet 118.

The cabinet 118 can be used for storage area for the laminar flow hood 100. For example, the cabinet can store the label printer 122, code scanner 124, a particulate counter, one or more cameras (e.g., preparation camera 115 and/or field-of-view camera 117), and/or any other feature. Any sensors (e.g., particulate counter) can partially extend through the enclosed housing 108 and/or be flush with the enclosed housing 108.

The cabinet 118 can be opened using the pull-out and/or slide-out hardware 132 (e.g., as illustrated in FIGS. 1D and 1E). In some cases, the cabinet 118 includes a door and hinge. A user 101 can pull the cabinet 118 to the open position to perform any necessary maintenance, stocking (e.g., filling the label printer 122 with labels), cleaning, and/or other activities that need to be performed. After performing these activities, a user 101 can push the cabinet 118 back to the closed position.

In some cases, the laminar flow hood 100 further includes a label printer 122 coupled to the computing device and positioned within the cabinet 118. The computing device causes the label printer 122 to print a label in response to an indication that a desired product is compounded. For example, a user 101 may provide the indication that the desired product is compounded by selecting and/or clicking on the display 106 that the desired product is compounded and the computing device can receive that indication and cause the label printer 122 to print the label for the desired product. In some cases, the enclosed housing 108 includes a label slot for the label to enter the enclosed housing 108 upon being printed. For example, the slot for the label to enter the enclosed housing 108 can be positioned where the label comes out from the label printer 122 when the cabinet 118 is in the closed position (e.g., as illustrated in FIG. 1C).

In some cases, the laminar flow hood 100 further includes a code scanner 124 coupled to the computing device and positioned within the cabinet 118. The computing device receives a code indicating an ingredient type in response to the code scanner 124 scanning a code. In some cases, the code is a barcode or QR code. For example, a user 101 may scan a code attached to packaging of an ingredient at the code scanner 124 and the computing device can receive the code indicated by the code of the ingredient type and/or quantity. In some cases, the computing device can cause the display 106 to indicate that the ingredient type and/or quantity is correct for the desired product. In some cases, the computing device can cause the display 106 to indicate that the ingredient type and/or quantity is incorrect for the desired product.

In some cases, the enclosed housing 108 includes a waste opening 126 and the laminar flow hood 100 further includes a waste chute 128 attached to an outside portion 130 of the enclosed housing 108 around the waste opening 126. In some cases, the laminar flow hood 100 further includes a waste sensor coupled to the computing device and a waste opening cover coupled to the computing device. In some cases, the waste sensor and the waste opening cover are coupled to the computing device, and a waste disposal indication from the user 101 that is captured by the waste sensor causes, via the computing device, the waste opening cover to open for the user 101 to dispose of waste. In some cases, the waste sensor is coupled to the waste opening cover, and a waste disposal indication from the user 101 that is captured by the waste sensor causes the waste opening cover to open for the user 101 to dispose of waste. This allows the user 101 to remove waste during the pharmaceutical compounding process without leaving the workspace surface 102. In some cases, the waste sensor is a motion sensor and the waster disposal indication is a motion by the user 101 that is captured by the waste sensor. In some cases, a servo motor is attached to the waste opening cover to physically move the waste opening cover to open upon receipt of the waste disposal indication of the user 101 (e.g., directly from the waste sensor or via the computing device).

FIG. 2 illustrates bottom view of a computing device mounted to a laminar flow hood. Referring to FIG. 2, a computing device 200 is attached to an underside 202 of a laminar flow hood by a computer mounting bracket 204. A gravimetric scale mounting bracket 206, which secures the gravimetric scale that includes a load cell to the workspace surface, is attached to the underside 202 of the laminar flow hood. The computing device 200 can include a plurality of ports 208 for connecting, for example using cords, with various features described herein, such as for example, a display, an air filtration system, one or more cameras, a gravimetric scale, a label printer, a code scanner, one or more sensors, one or more servo motors and/or any other features known to those having ordinary skill in the art of laminar flow hoods, biological safety cabinets, and/or gloveboxes that are compatible with a computing device. Although the plurality of ports 208 are illustrated for wired connection to the various features described herein, in some cases, one or more of the various features described herein may be wirelessly connected to the computing device.

In some cases, the gravimetric scale mounting bracket 206 may be mounted on each side (e.g., two or more sides of the gravimetric scale mounting bracket 206) to an underside 202 and/or frame of a laminar flow hood. In some cases, the computer mounting bracket 204 may be mounted on each side (e.g., two or more sides of the computer mounting bracket 204) to the underside 202 and/or frame of the laminar flow hood. The computer mounting bracket 204 and the gravimetric scale mounting bracket 206 may be mounted to the underside 202 and/or frame of the laminar flow hood by nut and bolt combinations or any other known ways of attaching a bracket to a frame and/or underside 202.

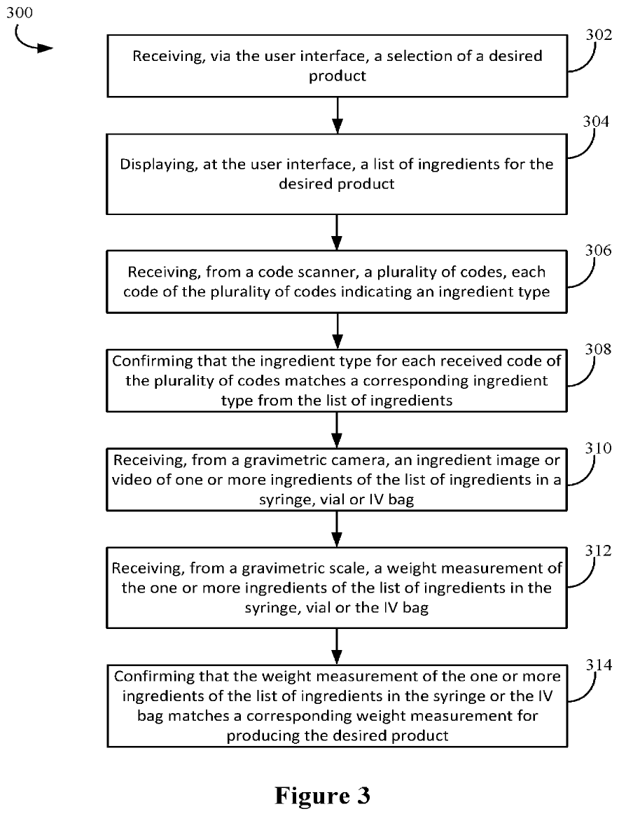

FIG. 3 illustrates a method of using a system for pharmaceutical compounding. Referring to FIG. 3, a method 300 of using a system (e.g., laminar flow hoods, biological safety cabinets, and/or gloveboxes) for pharmaceutical compounding includes receiving (302), via the user interface, a selection of a desired product. For example, a user may select a desired product, such as a drug, that the user wishes to manually compound by selecting a name of the desired drug from a list of drugs that can be manually compounded in the display.

The method 300 further includes displaying (304), at the user interface, a list of ingredients for the desired product, receiving (306), from a code scanner, a plurality of codes, with each code of the plurality of codes indicating an ingredient type, and confirming (308) that the ingredient type for each received code of the plurality of codes matches a corresponding ingredient type from the list of ingredients. For example, a user may gather the ingredients from the list of ingredients displayed (304) in the user interface and scan a code for each of the ingredients from the list of ingredients with a code scanner. Each code that is scanned can then be sent by the code scanner and received (306) by the computing device, which can confirm (308) that the ingredient type for each received code of the plurality of codes matches a corresponding ingredient type from the list of ingredients. In some cases, the confirmation that the ingredient type for a code can be displayed in the display as the correct ingredient and/or weight of the correct ingredient. In some cases, when a code received by the computing device cannot be confirmed because, for example, the code corresponds to a type of ingredient that is not on the list of ingredients for the desired product or the code includes a weight of a correct ingredient that does not correspond to the weight of that ingredient for the desired product, a rejection indication can be displayed in the user interface.

The method 300 further includes receiving (310), from a preparation camera an ingredient image or video of one or more ingredients of the list of ingredients in a syringe or IV bag, receiving (312), from a gravimetric scale, a weight measurement of the one or more ingredients of the list of ingredients in the syringe or the IV bag, and confirming (314) that the weight measurement of the one or more ingredients of the list of ingredients in the syringe or the IV bag matches a corresponding weight measurement for producing the desired product

In some cases, a picture or video of the one or more ingredients can be caused to be captured by the preparation camera and/or the field-of-view camera from a user pressing a foot pedal, which is sent to and received (310) by the computing device to be stored in storage for later viewing and confirmation that the one or more ingredients are correct and/or subsequently sent to an external source for viewing and confirmation that the one or more ingredients are correct. In some cases, the computing device receives (312) a weight measurement of the one or more ingredients from the gravimetric scale and, in response to receipt of the weight measurement, causes the preparation camera to capture a picture or video of the one or more ingredients on the gravimetric scale and sends that picture or video to the computing device (e.g., received (310) by the computing device) to be stored in storage for later viewing and confirmation that the one or more ingredients are correct and/or subsequently sent to an external source for viewing and confirmation that the one or more ingredients are correct. In some cases, the computing device receives (312) a weight measurement of the one or more ingredients from the gravimetric scale, confirms (314) that the weight measurement of the one or more ingredients of the list of ingredients in the syringe or the IV bag matches a corresponding weight measurement for producing the desired product and, in response to the confirmation that the weight measurement matches, causes the preparation camera to capture a picture or video of the one or more ingredients on the gravimetric scale and sends that picture or video to the computing device to be stored in storage for later viewing and confirmation that the one or more ingredients are correct and/or subsequently sent to an external source for viewing and confirmation that the one or more ingredients are correct. In some cases, the receiving (310), receiving (312), and confirming (314) occurs in that order. In some cases, the receiving (310), receiving (312), and confirming (314) includes these steps for each ingredient of the list of ingredients for the desired product.

In some cases, the method 300 further includes displaying, at the user interface, a confirmation indication that the weight measurement of one or more ingredients is correct in response to the weight measurement matching a corresponding weight measurement of the one or more ingredients. In some cases, the method 300 further includes displaying, at the user interface, a reject warning in response to the weight measurement of the one or more ingredients not matching a corresponding weight measurement of the one or more ingredients.

In some cases, the method 300 further includes receiving, from the preparation camera and/or the field-of-view camera, a compounded image of the desired product and receiving, from the gravimetric scale, a compounded weight measurement of the desired product. In some cases, the method 300 further includes confirming that the compounded weight measurement of the desired product matches a corresponding compounded weight measurement of the desired product. In some cases, the method 300 further includes receiving, from the field-of-view camera, an image or video of actions performed (e.g., compounding processes) by a user.

In some cases, a compounded picture or video of the desired product can be caused to be captured by the preparation camera and/or field-of-view camera from a user pressing a foot pedal, which is sent to and received by the computing device to be stored in storage for later viewing and confirmation that the desired product is correct and/or subsequently sent to an external source for viewing and confirmation that the desired product is correct. In some cases, the computing device receives a compounded weight measurement from the gravimetric scale and, in response to receipt of the compounded weight measurement, causes the preparation camera to capture a compounded picture or video of the desired product on the gravimetric scale and sends that compounded picture or video to the computing device (e.g., received (310) by the computing device) to be stored in storage for later viewing and confirmation that the desired product is correct and/or subsequently sent to an external source for viewing and confirmation that the desired product is correct. In some cases, the computing device receives a compounded weight measurement of the desired product from the gravimetric scale, confirms that the compounded weight measurement of the desired product in the syringe or the IV bag matches a corresponding compounded weight measurement of the desired product and, in response to the confirmation that the compounded weight measurement matches the corresponding compounded weight measurement of the desired product, causes the preparation camera to capture compounded a picture or video of the desired product on the gravimetric scale and sends that compounded picture or video to the computing device to be stored in storage for later viewing and confirmation that the desired product is correct and/or subsequently sent to an external source for viewing and confirmation that the desired product is correct.

In some cases, the method 300 further includes displaying, at the user interface, a confirmation indication that the compounded weight measurement of the desired product is correct in response to the compounded weight measurement matching a corresponding compounded weight measurement of the desired product. In some cases, the method 300 further includes displaying, at the user interface, a reject warning in response to the compounded weight measurement not matching a corresponding compounded weight measurement of the desired product.

In some cases, the method 300 further includes receiving an indication to activate the air filtration system and activating the air filtration system in response to the indication to activate the air filtration system. In some cases, the indication to activate the air filtration system includes a user pressing a button and/or switch on the overhead enclosure. In some cases, the indication to activate the air filtration system includes a user selecting an activate filtration system button and/or icon on the user interface. In some cases, the indication to activate the air filtration system includes receipt of a weight measurement from the gravimetric scale (of the compounded desired product and/or of the one or more ingredients). In some cases, the indication to activate the air filtration system includes receipt of an image or video from the preparation camera and/or the field-of-view camera (e.g., of the compounded desired product and/or of the one or more ingredients and/or an action performed by a user). In some cases, the indication to activate the air filtration system includes receipt of an indication from a motion sensor. In some cases, the indication to activate the air filtration system includes receipt of a measurement from a particulate counter, such as a number or range of particulates.

FIG. 4 illustrates a block diagram of components of a computing device for implementing at least some of the functionality of the pharmaceutical compounding system described herein, including the method 300 described with respect to FIG. 3. Referring to FIG. 4, the computing device 400 includes a processor 402, a storage 404 having instructions 406 stored thereon, and a plurality of input/output (I/O) interfaces 408 connected to the features of the pharmaceutical compounding system.

The processor 402 processes information received from the features and sends instructions to those features by way of the I/O interfaces 408 and processes data according to instructions stored in the storage 404, as well as executes instructions stored in the storage 404. Storage 404 can includes any computer readable storage media readable by the processor 402 and capable of storing software and/or instructions including the method 300 of FIG. 3. Storage 404 may include volatile and nonvolatile, removable and non-removable media implemented in any method or technology for storage of information. Examples of storage media include random access memory, read only memory, magnetic disks, optical disks, CDs, DVDs, flash memory, magnetic cassettes, magnetic tape, magnetic disk storage or other magnetic storage devices, or any other suitable non-transitory storage media. In some cases, the instructions can include certain aspects carried out by a pharmaceutical compounding application and/or software that renders user interfaces on a display 410. As used herein “storage media” do not consist of transitory, propagating waves. Instead, “storage media” refers to non-transitory media.

The I/O interfaces 408 may include wired or wireless connections to a display 410 (e.g., display 106 of FIGS. 1A-1F), an air filtration system 412, one or more cameras 414 (e.g., preparation camera 115 and/or field-of-view camera 117 of FIGS. 1A-1F), a gravimetric scale 416, a label printer 418, a code scanner 420, one or more sensors (e.g., particulate counter and/or motion sensor), one or more settings interfaces, one or more servo motors and/or any other features known to those having ordinary skill in the art of laminar flow hoods, biological safety cabinets, and/or gloveboxes that are compatible with a computing device.

In some cases, the display 410 may be a part of the computing device 400. The computing device 400 may also include additional output devices such as additional display screen(s), speakers, haptic devices for tactile feedback, and other types of output devices. In some cases, the display is a touchscreen, or touch-sensitive display which both depicts images and receives touch gesture input from the user. A touchscreen (which may be associated with or form part of the display 410) is an input device configured to detect the presence and location of a touch. The touchscreen may be a resistive touchscreen, a capacitive touchscreen, a surface acoustic wave touchscreen, an infrared touchscreen, an optical imaging touchscreen, a dispersive signal touchscreen, an acoustic pulse recognition touchscreen, or may utilize any other touchscreen technology. In some embodiments, the touchscreen is incorporated on top of a display 410 as a transparent layer to enable a user to use one or more touches to interact with objects or other information presented on the display 410.

This display 410 can include one or more devices such as, but not limited to, a liquid crystal display (LCD), a gas plasma-based flat panel displays, an organic light emitting diode (OLED) display, an electrophoretic ink (E ink) display, LCD projectors, or other types of display devices.

The computing device 400 may be configured to receive input from a user through, for example, a keyboard, mouse, trackpad, touch pad, touch screen, microphone, or other input device. The display 410 is configured to display one or more user interfaces (including the user interface described with respect to FIGS. 1A-1F) to the user.

In some cases, the user interface of the display 410 includes the use of a context menu, a menu within a menu bar, a menu item selected from a ribbon user interface, a graphical menu, and the like. Generally, the user interface is configured such that a user may easily interact with functionality of the compounding system. For example, a user may simply select (via, for example, touch, clicking, gesture or voice) an option within user interface to perform a process for pharmaceutical compounding.

In some cases, the computing device 400 can include wired or wireless interfaces communicating with the “outside world” (e.g., external networks), for example, to receive updates to pharmaceutical compounding processes and/or to send captured images or videos of the pharmaceutical compounding processes that occur in the pharmaceutical compounding system.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts that would be recognized by one skilled in the art are intended to be within the scope of the claims.

1. A system for pharmaceutical compounding, comprising:
a workspace surface having an outer boundary;
a display embedded within the workspace surface, the display having a plurality of edges that are impermeably sealed between the workspace surface, wherein the display is flush with the workspace surface;
a computing device providing a user interface to the display;
an enclosed housing extending from the outer boundary of the workspace surface and having an opening for a user to access the workspace surface; and
an air filtration system providing a negative pressure airflow within the enclosed housing.

2. The system of claim 1, further comprising a field-of-view camera positioned to capture images or video of an entirety of the workspace surface.

3. The system of claim 1, further comprising a gravimetric scale that includes a tray that is positioned above or flush with the workspace surface and a load cell positioned underneath the tray and the workspace surface.

4. The system of claim 3, further comprising a preparation camera positioned to capture images or video of the gravimetric scale.

5. The system of claim 4, further comprising a foot pedal coupled to the preparation camera and configured to cause the preparation camera to capture an image or video of the gravimetric scale when engaged by the user.

6. The system of claim 1, further comprising a cabinet attached to a first side of the enclosed housing.

7. The system of claim 6, further comprising a label printer coupled to the computing device and positioned within the cabinet,
wherein the computing device causes the label printer to print a label in response to an indication that a desired product is compounded.

8. The system of claim 7, wherein the enclosed housing comprises a label slot for the label to enter the enclosed housing upon being printed.

9. The system of claim 1, further comprising a code scanner coupled to the computing device,
wherein the computing device receives a code indicating an ingredient type in response to the code scanner scanning a code.

10. The system of claim 1, wherein the display is a touchscreen.

11. The system of claim 1, wherein the air filtration system comprises high efficiency particulate filters.

12. The system of claim 1, wherein the enclosed housing comprises a waste opening, the system further comprising:
a waste chute attached to an outside portion of the enclosed housing around the waste opening;
a waste sensor;
a waste opening cover coupled to the waste sensor and covering the waste opening;
wherein a waste disposal indication from the user that is captured by the waste sensor causes the waste opening cover to open for the user to dispose of waste.

13. (canceled)

14. The system of claim 1, wherein the enclosed housing further comprises medical grade plexiglass or medical grade stainless steel.

15. The system of claim 1, wherein the computing device comprises:
a processor;
a storage; and
instructions stored in the storage that when executed by the processor, cause the system to:
receive, via the user interface, a selection of a desired product;
display, at the user interface, a list of ingredients for the desired product;
receive, from a code scanner, a plurality of codes, each code of the plurality of codes indicating an ingredient type; and
confirm that the ingredient type for each received code of the plurality of codes matches a corresponding ingredient type from the list of ingredients.

16. The system of claim 15, wherein the instructions executed by the processor further direct the system to:
receive, from a preparation camera, a compounded image of the desired product; and
confirm that the desired product is correct from the compounded image.

17. The system of claim 15, wherein the instructions executed by the processor further direct the system to:
receive, from a gravimetric scale, a compounded weight measurement of the desired product; and
confirm that the compounded weight measurement of the desired product matches a corresponding compounded weight measurement of the desired product.

18. The system of claim 16, wherein the instructions executed by the processor further direct the system to send the compounded image of the desired product to an external source for confirmation that the desired product is correct.

19. The system of claim 17, wherein the instructions executed by the processor further direct the system to:
display, at the user interface, a reject warning in response to the compounded weight measurement not matching a corresponding compounded weight measurement of the desired product.

20. The system of claim 15, wherein the instructions executed by the processor further direct the system to:
receive an indication to activate the air filtration system; and
activate the air filtration system.

21. The system of claim 15, wherein the instructions executed by the processor further direct the system to:
receive, from a preparation camera, an ingredient image or video of one or more ingredients of the list of ingredients in a syringe or IV bag;
confirm that the one or more ingredients are correct from the ingredient image or video of the one or more ingredients of the list of ingredients in the syringe or IV bag.

22. The system of claim 15, wherein the instructions executed by the processor further direct the system to:
receive, from a gravimetric scale, a weight measurement of the one or more ingredients of the list of ingredients in the syringe or the IV bag; and
confirm that the weight measurement of the one or more ingredients of the list of ingredients in the syringe or the IV bag matches a corresponding weight measurement for producing the desired product.

23. The system of claim 15, wherein the instructions executed by the processor
receive user input using a microphone; and
output sound using a speaker.
